(19) United States
(12) Patent Application Publication
(10) Pub. No.: US 2021/0161880 A1
(43) Pub. Date: Jun. 3, 2021

(54) METHODS FOR TREATING DEMENTIA RELATED PSYCHOSIS

(71) Applicant: ACADIA Pharmaceuticals Inc., San Diego, CA (US)

(72) Inventor: Erin Foff, San Diego, CA (US)

(21) Appl. No.: 17/108,623

(22) Filed: Dec. 1, 2020

Related U.S. Application Data
(60) Provisional application No. 63/046,883, filed on Jul. 1, 2020.
(60) Provisional application No. 62/942,485, filed on Dec. 2, 2019.

(51) Int. Cl. A61K 31/4468; A61K 31/496; A61K 31/7048; A61K 31/343; A61K 31/137; A61K 31/138; A61K 31/4525; A61K 31/15; A61K 31/135; A61K 31/165; A61K 31/445; A61K 31/55; A61K 31/27; A61P 25/28; A61P 25/18

(52) U.S. Cl. CPC A61K 31/4468; A61P 25/18; A61K 31/7048; A61K 31/343; A61K 31/137; A61K 31/138; A61K 31/4525; A61K 31/15; A61K 31/135; A61K 31/165; A61K 31/445; A61K 31/55; A61K 31/27; A61P 25/28; A61K 31/496

(57) ABSTRACT

This disclosure provides, in part, a method of decreasing the risk of relapse in a patient having dementia related psychosis, comprising administering to the patient an effective amount of pimavanserin daily. Also provided herein is a method of treating a dementia related psychosis in a patient in need thereof, comprising administering an effective amount of pimavanserin daily to the patient.

### CROSS REFERENCE TO RELATED APPLICATIONS

This application claims priority to and the benefit of U.S. Provisional Patent Application No. 62/942,485, filed on Dec. 2, 2019 and U.S. Provisional Patent Application No. 63/046,883, filed on Jul. 1, 2020, each of which is incorporated herein by reference in its entirety.

### BACKGROUND

Dementia is a syndrome with core symptoms, most notably a decline in cognitive function, but with a variety of other manifestations that are rarely confined to one subtype. Common subtypes of dementia include Alzheimer's disease (AD), vascular dementia (VaD), Parkinson's disease dementia (PDD), dementia with Lewy bodies (DLB), and the frontotemporal dementia (FTD)-spectrum disorders (including behavioral variant FTD), progressive supranuclear palsy (PSP), and corticobasal degeneration (CBD)).

About 30% of dementia patients will experience psychosis. Psychosis is associated with adverse patient outcomes and increased cost of care. Use of typical or atypical antipsychotics have shown none to modest efficacy and poses significant safety concerns including: increased risk of mortality in elderly patients with dementia related psychosis, risk of acceleration of cognitive decline, and numerous toxicities associated with varied receptor activities. In fact, anti-psychotics are generally are associated with increased risk of death, and are not approved for elderly patients with dementia related psychosis. Additionally, there is often concern that such anti-psychotics can exacerbate cognition and thus lead to or add to cognition decline. Thus, treatment of dementia related psychosis represents an area of high unmet need, especially a treatment that may be administered substantially long term and/or can significantly minimize the risk of any additional psychotic episodes.

N-(4-Fluorophenylmethyl)-N-(1-methylpiperidin-4-yl)-N′-(4-(2-methylpropyloxy)phenylmethyl)carbamide), also known as pimavanserin (or NUPLAZID®), is a selective serotonin 5-HT2A inverse agonist and has been approved by U.S. Food and Drug Administration (FDA) in treating hallucinations and delusions associated with Parkinson's disease.

### SUMMARY

This disclosure provides, in part, a method of decreasing the risk of relapse in a patient having dementia related psychosis, comprising administering to the patient an effective amount of pimavanserin or a pharmaceutically acceptable salt thereof.

In one aspect, provided herein is a method of decreasing the risk of relapse in a patient having dementia related psychosis, comprising administering to the patient an effective amount of pimavanserin or a pharmaceutically acceptable salt, daily, wherein relapse is indicated by a) at least 15% increase of psychosis as measured by a SAPS-H+D Total Score in the patient and/or b) a CGI-I score of 3 or more after 12 weeks or more of administration of the pimavanserin.

In certain embodiments, the relapse is indicated by a) at least 30% increase of psychosis as measured by a SAPS-H+D Total Score in the patient and/or b) a CGI-I score of 6 or more after 12 weeks or more of administration of the pimavanserin.

In some embodiments, the contemplated patient may have moderate to severe psychosis before administration of pimavanserin, as measured by a SAPS-H+D total score of 10 more, a SAPS-H+D global item H7 or D13 score of 4 or more, and a CGI-S score of 4 or more.

Also provided herein is a method of treating a dementia related psychosis in a patient in need thereof, comprising administering an effective amount of pimavanserin daily to the patient.

Contemplated methods include a method of treating a dementia related psychosis in a patient in need thereof, comprising administering an effective amount of pimavanserin daily to the patient, wherein the patient has at least at 30% reduction on SAPS-H+D total score and/or a CGI score of 1 or 2 after 4 weeks or more of administration as compared to a baseline before administration.

For example, the dementia related psychosis may one of Alzheimer's disease dementia, dementia with Lewy Bodies, Parkinson's disease dementia, vascular dementia, and frontotemporal dementia.

Another contemplated method provided herein is a method of treating psychosis associated with vascular or frontotemporal dementia in a patient in need thereof, comprising administered an effective amount of pimavanserin daily to the patient.

Also provided herein is a method of decreasing the risk of relapse in a patient having dementia related psychosis, comprising administering to the patient an effective amount of pimavanserin or a pharmaceutically acceptable salt, daily, wherein relapse is indicated by a) a 30% increase of psychosis as measured by a SAPS-H+D Total Score in the patient and/or b) a CGI-I score of 6 or more after 12 weeks or more of administration of the pimavanserin.

The effective amount of contemplated methods disclosed herein may be, for example, a daily dose of pimavanserin of 20 mg, 34 mg, or 40 mg. In certain embodiments, the effective amount is a daily dose of pimavanserin of 34 mg, provided in one fixed dose or multiple fixed doses.

In another aspect, the present disclosure provides a method of reducing the risk of exacerbation of psychosis secondary to neurodegenerative or vascular disorders in a patient in need thereof, comprising administering an effective amount of pimavanserin to the patient.

The contemplated neurodegenerative or vascular disorder may be Alzheimer's disease, dementia with Lewy Bodies, Parkinson's disease, vascular dementia, or frontotemporal dementia.

Another contemplated method provided herein is a method of improving the response in a patient having dementia related psychosis, comprising administering to the patient an effective amount of pimavanserin or a pharmaceutically acceptable salt, once daily, wherein the reduction in total SAPS-H+D score in week 2 is better than before administration, and wherein the reduction in SAPS-H+D is maintained or further reduced in week 12.

In another aspect, provided herein is a method of stabilizing a patient having dementia related psychosis, comprising administering to the patient an effective amount of pimavanserin or a pharmaceutically acceptable salt, once daily, wherein the reduction in total SAPS-H+D score in week 2 is better than before administration, and wherein the reduction in SAPS-H+D is maintained or further reduced in week 12.

In certain embodiments, the effective amount may be a daily dose of pimavanserin of 20 mg, 34 mg, or 40 mg. In some embodiments, the effective amount is a daily dose of pimavanserin of 34 mg, provided in one fixed dose or multiple fixed doses.

Also provided herein is a method of treating a patient with a recent history of psychosis, comprising administering an effective amount of pimavanserin to the patient.

### BRIEF DESCRIPTION OF THE DRAWINGS

FIG. 1 shows the total SAPS-H+D change in subjects treated with pimavanserin during the open-label period.

FIG. 2A shows the percentage of subjects meeting the response criteria at Week 2 and Week 4 during the open-label period. FIG. 2B shows the percentage of subjects meeting the response criteria at Weeks 2, 4, and 8 during the open-label period. FIG. 2C shows the percentage of subjects meeting the response criteria across dementia subtypes at Weeks 2, 4, and 8 during the open-label period. FIG. 2D shows the percentage of subjects meeting the response criteria across baseline MMSE at Weeks 2, 4, and 8 during the open-label period. FIG. 2E shows the percentage of subjects meeting the response criteria across age at Weeks 2, 4, and 8 during the open-label period. FIG. 2F shows the percentage of subjects meeting the response criteria across psychosis severity at Weeks 2, 4, and 8 during the open-label period.

FIG. 3 shows the percentage of subjects meeting the SAPS-H+D category during the open-label period.

FIG. 4A shows the total SAPS-H+D change in subjects treated with pimavanserin across subtypes of dementia. FIG. 4B shows the total SAPS-H+D change in subjects treated with pimavanserin across baseline MMSE. FIG. 4C shows the total SAPS-H+D change in subjects treated with pimavanserin across baseline dementia severity.

FIG. 5A shows CGI-S score change from baseline in subjects treated with pimavanserin (overall population). FIG. 5B shows CGI-S score change from baseline in subjects treated with pimavanserin across subtypes of dementia.

FIG. 6 shows the probability of relapse of dementia related psychosis in pimavanserin or placebo-treated patients during the double-blind period.

FIG. 7 shows the probability of risk of all-cause discontinuation in pimavanserin or placebo-treated patients during the double-blind period.

FIG. 8 shows the MMSE score change from baseline through Week 46.

### DETAILED DESCRIPTION

The features and other details of the disclosure will now be more particularly described. Before further description of the present disclosure, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

### Definitions

“Treating” includes any effect, e.g., lessening, reducing, modulating, or eliminating, that results in the improvement of the condition, disease, disorder and the like.

“Individual,” “patient,” or “subject” are used interchangeably and include any animal, including mammals, preferably mice, rats, other rodents, rabbits, dogs, cats, swine, cattle, sheep, horses, or primates, and most preferably humans. The compounds of the disclosure can be administered to a mammal, such as a human, but can also be other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, and the like).

The term “pharmaceutically acceptable carrier” or “pharmaceutically acceptable excipient” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutical composition” as used herein refers to a composition comprising at least one compound as disclosed herein formulated together with one or more pharmaceutically acceptable carriers.

In the present specification, the term “therapeutically effective amount” means the amount of the subject compound that will elicit the biological or medical response of a tissue, system, animal or human that is being sought by the researcher, veterinarian, medical doctor or other clinician. The compounds of the disclosure are administered in therapeutically effective amounts to treat a disease. Alternatively, a therapeutically effective amount of a compound is the quantity required to achieve a desired therapeutic and/or prophylactic effect, such as an amount which results in the treatment of dementia related psychosis.

“Mini-Mental State Examination” (MMSE) is a brief 30-point questionnaire that is used to quantitatively assess cognition (Folstein et al. J Psychiatr Res. 1975; 12(3): 189-198). The MMSE includes simple questions and problems in a number of areas: the time and place of testing, repeating lists of words, arithmetic, language use and comprehension, and copying a drawing.

“Scale for the Assessment of Positive Symptoms” (SAPS) is designed to measure positive psychotic symptoms (Andreasen N. Scale for the Assessment of Positive Symptoms (SAPS). Iowa City, Iowa: University of Iowa, 1984). Positive symptoms include hallucinations, delusions, abnormalities in language and behavior, and disordered thought processes. “SAPS Hallucinations and Delusions” (SAPS-H+D) subscales consist of 20 items, including 2 global ratings of severity for hallucinations (H7) and delusions (D13).

“Clinical Global Impression (CGI)” scale is a clinician-rated, 7-point scale that is designed to rate the severity of the subject's depression at the time of assessment using the Investigator's judgment and past experience with subjects who have the same disorder. “Clinical Global Impression-Improvement (CGI-I)” is a clinician-rated, 7-point scale that is designed to rate the improvement in the subject's depression at the time of assessment, relative to the symptoms at Baseline. “Clinical Global Impression-Severity (CGI-S)” is an assessment of the current severity of the patient's disease.

“Zarit Burden Interview” (ZBI) assesses the stress experienced by caregivers of patients with dementia (Zarit et al. Gerontologist. 1980; 20(6): 649-655). The ZBI consists of 22 statements reflecting how people sometimes feel when taking care of another person. The statements are phrased as questions for the family member study partner/caregiver to indicate how often they feel the way described in the statement. Responses are Never, Rarely, Sometimes, Quite Frequently, and Nearly Always.

“EuroQol-5 dimensions-5 level” (EQ-5D-5L) is a standardized instrument used as a measure of health outcome. It measures 5 dimensions (mobility, self-care, usual activities, pain/discomfort, anxiety/depression) each of which has 5 potential responses. The responses record 5 levels of severity (no problems/slight problems/moderate problems/severe problems/extreme problems) within a particular EQ-5D dimension. In some embodiments, the EQ-5D-5L is EQ-5D-5L proxy version 1, where a study partner/caregiver (the proxy) is asked to rate subject's health-related quality of life in their (the proxy's) opinion.

“Karolinska Sleepiness Scale” (KSS) is a 9-item, self-reported subjective measure of a subject's level of drowsiness (Akerstedt et al. Int J Neurosci. 1990; 52(1-2): 29-37).

### Methods

The disclosure provides, in part, method of decreasing the risk of relapse in a patient having dementia related psychosis, comprising administering to the patient an effective amount of pimavanserin or a pharmaceutically acceptable salt, daily, wherein relapse is indicated by a) at least 15% increase of psychosis as measured by a SAPS-H+D Total Score in the patient and/or b) a CGI-I score of 3 or more after 12 weeks or more of administration of the pimavanserin.

In certain embodiments, the relapse is indicated by a) at least 30% increase of psychosis as measured by a SAPS-H+D Total Score in the patient and/or b) a CGI-I score of 6 or more after 12 weeks or more of administration of the pimavanserin.

In some embodiments, the contemplated patient may have moderate to severe psychosis before administration of pimavanserin, as measured by a SAPS-H+D total score of 10 more, a SAPS-H+D global item H7 or D13 score of 4 or more, and a CGI-S score of 4 or more.

For example, relapse may be indicated by a) a 30% increase of psychosis as measured by a SAPS-H+D Total Score in the contemplated patient and b) a CGI-I score of 6 or more after 38 weeks of administration of the pimavanserin.

The contemplated patient may have at least a 50% reduction in psychosis symptoms as measured by the SAPS-H+D Total Score at 12 weeks or more of administration as compared to psychosis symptoms before administration. In certain embodiments, the contemplated patient may have at least a 70% reduction in psychosis symptoms as measured by the SAPS-H+D Total Score at 12 weeks or more of administration as compared to psychosis symptoms before administration. In other embodiments, the contemplated patient may have at least 55%, 60%, 65%, 75%, or 80% reduction in psychosis symptoms as measured by the SAPS-H+D Total Score at 12 weeks, 18 week, 24 weeks, 30 weeks or more of administration.

For example, the risk of relapse in the contemplated patient may be reduced by at least half. As another example, the risk of relapse in the contemplated patient may be reduced by at least 2.5 times. As another example, the risk of relapse in the contemplated patient may be reduced by at least 2.8 times.

In other embodiments, the contemplated patient has at least at 30% reduction on SAPS-H+D total score and/or a CGI score of 1 or 2 after 4 weeks or more of administration as compared to a baseline before administration.

Also provided herein is a method of treating a dementia related psychosis in a patient in need thereof, comprising administering an effective amount of pimavanserin daily to the patient, wherein the patient has at least at 30% reduction on SAPS-H+D total score and/or a CGI score of 1 or 2 after 4 weeks or more of administration as compared to a baseline before administration.

In certain embodiments, after at least 4 weeks or more of administration, the risk of discontinuation of administration due to relapse or other adverse event in the contemplated patient is reduced by at least half compared to a patient not taking pimavanserin.

For example, over 9 months or more of administration, the contemplated patient may not suffer from substantial cognitive impairment compared to before administration, as measured by a MMSE score. In another example, after 9 months or more of administration, the contemplated patient may not suffer from substantial cognitive impairment compared to before administration, as measured by a MMSE score.

In certain methods provided herein, the dementia related psychosis may be one of Alzheimer's disease dementia, dementia with Lewy Bodies, Parkinson's disease dementia, vascular dementia, or frontotemporal dementia. In some embodiments, the dementia related psychosis is dementia with hallucinations and/or delusions, for example hallucinations and delusions associated with Parkinson's or Alzheimer's disease. In other embodiments, contemplated methods of treating dementia related psychosis does not include treating psychosis associated with Parkinson's and/or Alzheimer's disease and/or dementia with Lewy Bodies.

The contemplated methods provided herein include a method of treating psychosis associated with vascular or frontotemporal dementia in a patient in need thereof, comprising administered an effective amount of pimavanserin daily to the patient.

Another contemplated method described herein is a method of decreasing the risk of relapse in a patient having dementia related psychosis, comprising administering to the patient an effective amount of pimavanserin or a pharmaceutically acceptable salt, daily, wherein relapse is indicated by a) a 30% increase of psychosis as measured by a SAPS-H+D Total Score in the patient and/or b) a CGI-I score of 6 or more after 12 weeks or more of administration of the pimavanserin.

In certain embodiments of methods provided herein, the effective amount may be a daily dose of pimavanserin of 20 mg, 34 mg, or 40 mg. For example, the effective amount may be a daily dose of pimavanserin of about 5 mg to about 40 mg, based on the free base form, of N-(4-fluorophenylmethyl)-N-(1-methylpiperidin-4-yl)-N′-(4-(2-methylpropyloxy)phenylmethyl)carbamide. In some embodiments, the effective amount is a daily dose of pimavanserin of 34 mg, provided in one fixed dose or multiple fixed doses.

The contemplated patient may be concurrently administered a strong CYP3A4 inhibitor and the effective amount is a 10 mg daily dose of pimavanserin. Examples of CYP3A4 inhibitor include ketoconazole, clarithromycin, indinavir, and itraconazole.

In some embodiments, the pimavanserin is administered as a tartrate salt.

In certain embodiments, the contemplated patient may be also administered a SSRI or a SNRI. Examples of SSRI or SNRI is selected from the group consisting of citalopram, desmethylcitalopram, didesmethylcitalopram, desvenlafaxine, escitalopram (LEXAPRO), fluoxetine (PROZAC), fluvoxamine, paroxetine (PAXIL), sertraline, vilazodone, vortioxetine, desvenlafaxine, duloxetine, levomilnacipran, sibutramine, tramadol, milnacipran, norsertraline, serproxetin, and venlafaxine.

In other embodiments, the methods provided herein further comprises administering an anti-dementia agent to the patient. A contemplated anti-dementia agent is a cholinesterase inhibitor. For example, anti-dementia agents include donepezil, galantamine, and rivastigmine.

In another aspect, provided herein is a method of reducing the risk of exacerbation of psychosis secondary to neurodegenerative or vascular disorders in a patient in need thereof, comprising administering an effective amount of pimavanserin to the patient.

In some embodiments, the neurodegenerative or vascular disorder may be Alzheimer's disease, dementia with Lewy Bodies, Parkinson's disease, vascular dementia, or frontotemporal dementia.

In certain embodiments, after 4 weeks or more of administration, the risk of exacerbation of psychosis in the contemplated patient is reduced by at least half compared to a patient not taking pimavanserin.

As contemplated herein, exacerbation does not include acute exacerbation of psychosis. Contemplated patients, in some embodiments, are not administered risperidone (or tacrine).

Also provided herein is a method of improving the response in a patient having dementia related psychosis, comprising administering to the patient an effective amount of pimavanserin or a pharmaceutically acceptable salt, once daily, wherein the reduction in total SAPS-H+D score in week 2 is better than before administration, and wherein the reduction in SAPS-H+D is maintained or further reduced in week 12.

A contemplated method provided herein also includes a method of stabilizing a patient having dementia related psychosis, comprising administering to the patient an effective amount of pimavanserin or a pharmaceutically acceptable salt, once daily, wherein the reduction in total SAPS-H+D score in week 2 is better than before administration, and wherein the reduction in SAPS-H+D is maintained or further reduced in week 12.

In certain embodiments of methods provided herein, the effective amount may be a daily dose of pimavanserin of 20 mg, 34 mg, or 40 mg. For example, the effective amount may be a daily dose of pimavanserin of about 5 mg to about 40 mg, based on the free base form, of N-(4-fluorophenylmethyl)-N-(1-methylpiperidin-4-yl)-N′-(4-(2-methylpropyloxy)phenylmethyl)carbamide. In some embodiments, the effective amount is a daily dose of pimavanserin of 34 mg, provided in one fixed dose or multiple fixed doses.

The contemplated patient may also concomitantly be taking a strong CYP3A4 inhibitor and the effective amount is a 10 mg daily dose of pimavanserin. Examples of CYP3A4 inhibitor include ketoconazole, clarithromycin, indinavir, and itraconazole.

In certain embodiments, the pimavanserin is administered as a tartrate salt.

Also provided herein is a method of treating a patient with a recent history of psychosis, comprising administering an effective amount of pimavanserin to the patient.

Unless otherwise indicated, a subject can be a mammal, a primate, a monkey or a human. In certain embodiments of the methods provided herein, the subject is a human subject.

In some embodiments, the patient is a human subject. For example, the contemplated patient may be a male patient. For example, the contemplated patient may be a female patient.

The age of the contemplated human subject is 50 years or older, or 55 years or old, or 60 years or older, or 65 years or older, or 70 years or older at baseline.

In other embodiments, N-(4-fluorophenylmethyl)-N-(1-methylpiperidin-4-yl)-N′-(4-(2-methylpropyloxy)phenylmethyl)carbamide may be administered in the form of a tablet. In some embodiments, N-(4-fluorophenylmethyl)-N-(1-methylpiperidin-4-yl)-N′-(4-(2-methylpropyloxy)phenylmethyl)carbamide may be administered in the form of a capsule.

In certain embodiments, N-(4-fluorophenylmethyl)-N-(1-methylpiperidin-4-yl)-N′-(4-(2-methylpropyloxy)phenylmethyl)carbamide may be administered each day for at least 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, or 3 months, 6 months or more.

In some embodiments, N-(4-fluorophenylmethyl)-N-(1-methylpiperidin-4-yl)-N′-(4-(2-methylpropyloxy)phenylmethyl)carbamide may be administered in the form of a tartrate salt. In some embodiments, the 34 mg may be administered in one capsule or two tablets (2×17 mg tablets). In other embodiments, the 20 mg may be administered in one capsule or two tablets (2×10 mg tablets). In certain embodiments, the patient is concurrently being administered a CYP3A4 inhibitor, wherein orally administering comprises administering 10 mg of N-(4-fluorophenylmethyl)-N-(1-methylpiperidin-4-yl)-N′-(4-(2-methylpropyloxy)phenylmethyl)carbamide daily.

In certain embodiments of methods provided herein, pimavanserin or pharmaceutically acceptable salt thereof may be administered orally to the human subject. For example, a tartrate salt of pimavanserin may be administered to the human subject.

Pimavanserin or pharmaceutically acceptable salt thereof can, for example, be administered once, twice or three times per day. Pimavanserin or pharmaceutically acceptable salt thereof can, for example, be administered in a daily dose between 0.5 mg to 120 mg, or between 8 mg to 42 mg. For example, the daily dose of pimavanserin may be 20 mg, 34 mg, or 40 mg daily. In some embodiments, the effective amount is a daily dose of pimavanserin of 34 mg, provided in one fixed dose or multiple fixed doses.

### Compound

N-(4-Fluorophenylmethyl)-N-(1-methylpiperidin-4-yl)-N′-(4-(2-methylpropyloxy)phenylmethyl)carbamide is also known as pimavanserin; N-[(4-fluorophenyl)methyl]-N-(1-methyl-4-piperidinyl)-N′-[[4-(2-methylpropoxy)phenyl]methyl]-urea, 1-(4-fluorobenzyl)-1-(1-methylpiperidin-4-yl)-3-[4-(2-methylpropoxy)benzyl]urea or ACP-103 and is represented by the chemical formula:

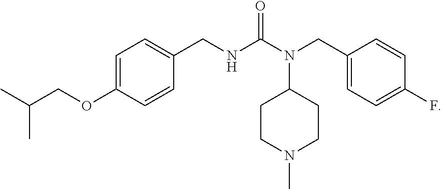 [SMILES: CC(C)COc1ccc(CNC(=O)N(Cc2ccc(F)cc2)C2CCN(C)CC2)cc1]

N-(4-Fluorophenylmethyl)-N-(1-methylpiperidin-4-yl)-N′-(4-(2-methylpropyloxy)phenylmethyl)carbamide maybe administered as a tartrate salt, which is urea, N-[(4-fluorophenyl)methyl]-N-(1-methyl-4-piperidinyl)-N′-[[4-(2-methylpropoxy)phenyl]methyl]-,(2R,3R)-2,3-dihydroxybutanedioate (2:1), and represented by the chemical formula:

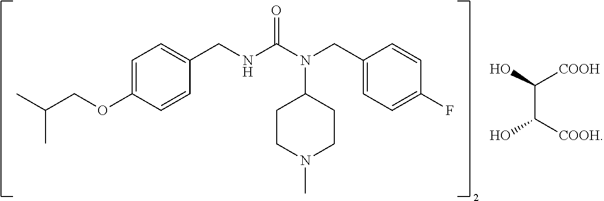

Pimavanserin (i.e., N-(4-Fluorophenylmethyl)-N-(1-methylpiperidin-4-yl)-N′-(4-(2-methylpropyloxy)phenylmethyl)carbamide) can be synthesized according to the method disclosed in Scheme I.

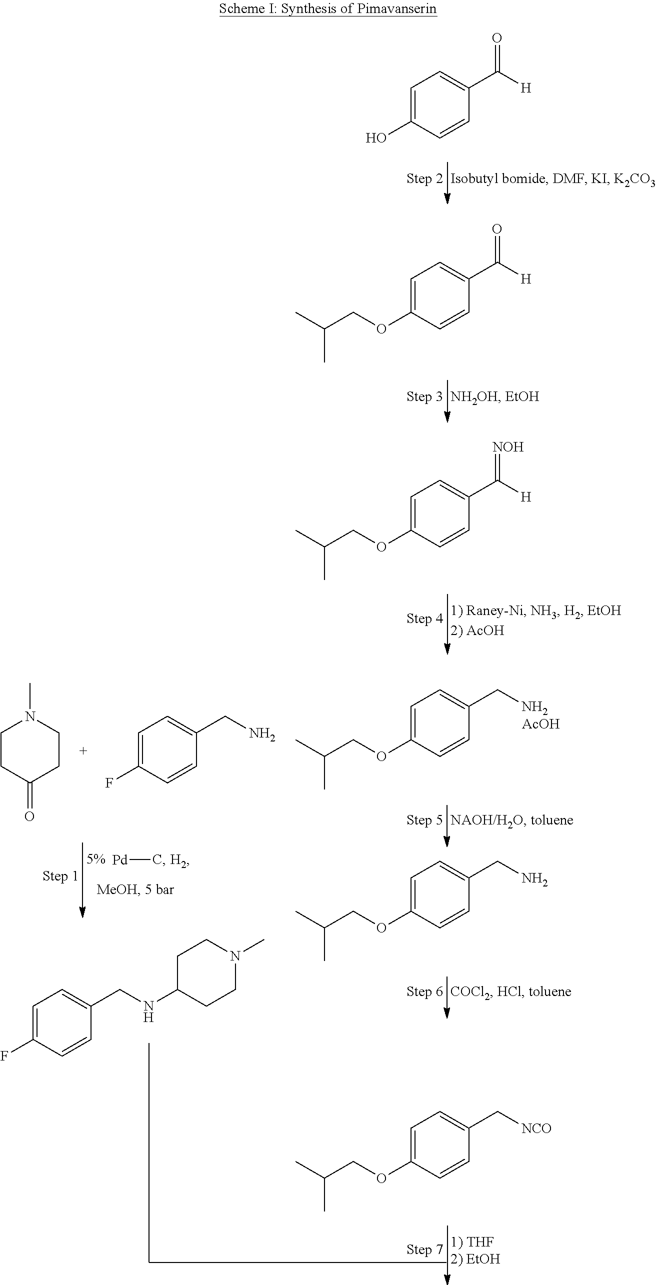

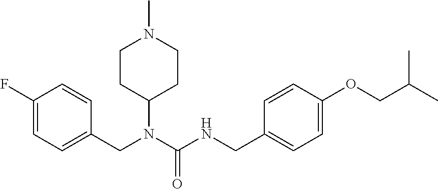 [SMILES: CC(C)COc1ccc(CNC(=O)N(Cc2ccc(F)cc2)C2CCN(C)CC2)cc1]

Pimavanserin can be obtained in a number of salt and crystalline forms. Exemplary pharmaceutically acceptable salts include the tartrate, hemi-tartrate, citrate, fumarate, maleate, malate, phosphate, succinate, sulphate, and edisylate (ethanedisulfonate) salts. Pimavanserin, and salts thereof including the aforementioned ions, among others, are described, for example, in International Patent Publication WO 2008/144326, U.S. Patent Publication Nos. 2006-0111399 and 2010-0305329, and International Patent Application WO2017015272, the entirety of which is incorporated herein by reference. In an embodiment provided herein, pimavanserin is the tartrate salt of pimavanserin. Several crystalline forms of the tartrate salt of pimavanserin are known. In an embodiment provided herein, pimavanserin is the crystalline form of the tartrate salt of pimavanserin Form A. In another embodiment, pimavanserin is the crystalline form of the tartrate salt of pimavanserin Form C.

In certain embodiments of the methods provided herein, pimavanserin or a pharmaceutically acceptable salt thereof is administered to the subject in an oral dosage form, such as, for example, a tablet, a capsule, or a lozenge and the like.

In some embodiments, the drug product is formulated with standard pharmaceutical excipients at a 34 mg pimavanserin (40 mg of pimavanserin tartrate) in capsules for oral administration. In certain embodiments the capsule formulation includes inactive ingredients magnesium stearate and microcrystalline cellulose. The following inactive ingredients can, for example, be present as components of the capsule shell: black iron oxide, FD&C blue #1, hypromellose, titanium dioxide, and yellow iron oxide.

In certain embodiments of the methods provided herein, pimavanserin or a pharmaceutically acceptable salt thereof is orally administered to the subject in a capsule, wherein the amount of pimavanserin or pharmaceutically acceptable salt in the capsule is between 2 mg to 80 mg, between 5 mg to 45 mg, or between 9 mg to 42 mg.

In certain embodiments of the methods provided herein, pimavanserin or a pharmaceutically acceptable salt thereof is orally administered to the subject in a tablet, wherein the amount of pimavanserin or pharmaceutically acceptable salt in the tablet is between 2 mg to 80 mg, between 5 mg to 45 mg, or between 9 mg to 42 mg.

Disclosed method include administration of N-(4-fluorophenylmethyl)-N-(1-methylpiperidin-4-yl)-N′-(4-(2-methylpropyloxy)phenylmethyl)carbamide orally by table or capsule (34 mg of compounds). Tablets may contain 20 mg of pimavanserin tartrate, which is equivalent to 17 mg of pimavanserin free base, or 11.8 mg of pimavanserin tartrate, which is equivalent to 10 mg of pimavanserin free base. In the above formulations, pimavanserin is in crystalline and/or amorphous form.

Pimavanserin (i.e., N-(4-fluorophenylmethyl)-N-(1-methylpiperidin-4-yl)-N′-(4-(2-methylpropyloxy)phenylmethyl)carbamide) or a pharmaceutically acceptable salt thereof (e.g., tartrate salt) may be administered in the form of a capsule, e.g. a capsule may contain 40 mg of pimavanserin tartrate, which is equivalent to 34 mg of pimavanserin free base, or 20 mg of pimavanserin tartrate, which is equivalent to 17 mg of pimavanserin free base, or 11.8 mg of pimavanserin tartrate, which is equivalent to 10 mg of pimavanserin free base.

In some embodiments, the drug product is formulated with standard pharmaceutical excipients at a 17 mg strength (20 mg of pimavanserin tartrate) in immediate-release tablets for once-daily oral administration.

In some embodiments, the drug product is formulated with standard pharmaceutical excipients at a 10 mg strength (11.8 mg of pimavanserin tartrate) in immediate-release tablets for once-daily or twice-daily oral administration.

Pimavanserin once daily a 10 mg tablet can, for instance, be administered to the subject when a strong CYP3A4 inhibitor (e.g., ketoconazole) is also being administered to the subject.

In some embodiments, the dose for the indication of adjunctive treatment of MDD indication is 34 mg pimavanserin taken orally as two 17 mg tablets once daily.

In some embodiments, the dose and pharmaceutical composition of pimavanserin are those disclosed in International Application No. PCT/US2018/048096, which is incorporated herein for all purposes. Pimavaserin can be in a crystalline form. Such forms have been described as Forms A, B, C, D, etc., e.g. in WO 2006/037043, WO 2007/133802 and WO 2008/144326.

In some embodiments, the pharmaceutical composition comprises granulated pimavanserin tartrate, Form C which may include a small percentage of Form A, including a pharmaceutically acceptable diluent, binder or excipient, or combination thereof.

In some embodiments, the pharmaceutical compositions are provided as a two-piece hard shell capsules made of gelatin (fish, mammalian, or vegetable sourced) or other combinations. The two-piece hard shell capsules may contain the pimavanserin granules with a filler/diluent and/or lubricants. In some embodiments, the capsules are size 3 or 4 capsules. In some embodiments, the capsules are size 4 capsules. In some embodiment, the capsules are two-piece capsules of gelatin or hydroxypropyl methylcellulose (HPMC). Some commercial examples are VCAPS®, VCAPS® PLUS, CONI-SNAP® capsules marketed by Capsugel.

In some embodiments, the doses referred to herein refers to pimavanserin free base. In some embodiments, the doses referred to herein refers to pimavanserin tartrate Form C (e.g., 40 mg of pimavanserin tartrate, equivalent to 34 mg pimavanserin free base). In some embodiments, the doses refer to pimavanserin tartrate Form C encapsulated in capsules of size 3 or 4, such as capsules of size 4.

Provided are also embodiments wherein pimavanserin (granulated), microcrystalline cellulose, for example Avicel PH302 or an equivalent microcrystalline cellulose, and/or magnesium stearate, for example vegetable grade are encapsulated in a capsule of size 4, for example a two-piece capsule.

One embodiment of the compositions described herein includes pimavanserin tartrate granulation without binder, dried, and thereafter blended with less than 60% w/w microcrystalline cellulose such as Avicel PH302 or equivalent microcrystalline cellulose, and about 1% w/w magnesium stearate.

In some embodiments the compositions described herein comprise granulated pimavanserin and microcrystalline cellulose is at least 20% w/w microcrystalline cellulose, such as 30% w/w microcrystalline cellulose, such as 40% w/w microcrystalline cellulose, such as 50% w/w microcrystalline cellulose, such as 50-89% w/w microcrystalline cellulose, such as 20-94% w/w, such as 50-94% w/w, such as 57-94% w/w, such as 57-89% w/w microcrystalline cellulose, such as 57-79% w/w microcrystalline cellulose, or 57-60% w/w microcrystalline cellulose, or 57-59.5% w/w microcrystalline cellulose, or 58.5-59.5% w/w microcrystalline cellulose, or 59% w/w microcrystalline cellulose.

In some embodiments the compositions described herein comprise granulated pimavanserin and microcrystalline cellulose and magnesium stearate, such as 0.1-3% w/w, such as 0.5-2% w/w magnesium stearate, or 0.5-1.5% w/w magnesium stearate, or 1% w/w magnesium stearate.

In some embodiments the compositions described herein comprise granulated pimavanserin (5, 10, 20 or 34 mg) and microcrystalline cellulose is at least 20% w/w microcrystalline cellulose, such as 30% w/w microcrystalline cellulose, such as 40% w/w microcrystalline cellulose, such as 50% w/w microcrystalline cellulose, such as 50-89% w/w microcrystalline cellulose, such as 20-94% w/w, such as 50-94% w/w, such as 57-94% w/w, such as 57-89% w/w microcrystalline cellulose, such as 57-79% w/w microcrystalline cellulose, or 57-60% w/w microcrystalline cellulose, or 57-59.5% w/w microcrystalline cellulose, or 58.5-59.5% w/w microcrystalline cellulose, or 59% w/w microcrystalline cellulose and magnesium stearate, such as 0.1-3% w/w, such as 0.5-2% w/w magnesium stearate, or 0.5-1.5% w/w magnesium stearate, or 1% w/w magnesium stearate.

The compositions disclosed herein comprise pimavanserin and additional compatible excipients, e.g. sugars, sucrose, mannitol, sorbitol, polysaccharides (e.g. from corn, wheat, rice, potato), as well as pregelatinized or partially pregelatinized starches (e.g. STARCH 1500®), cellulose preparations such as microcrystalline cellulose (MCC) (e.g. AVICEL® PH 302, AVICEL® PH 102, VIVAPUR®302, VIVAPUR®102, EMCOCEL® HD 90), silicified microcrystalline cellulose (e.g. PROSOLV®50, PROSOLV®90, PROSOLV® HD90), lactose cellulose blends (e.g. CELLATOSE®80, CELLATOSE®90, PROSOLV® EASYtab SP), hydroxypropylmethyl cellulose, hydroxymethyl cellulose, polyvinylpyrrolidone, lubricants such as magnesium stearate, sodium stearyl fumarate, colloidal silicon dioxide, and talc.

Provided are also embodiments wherein 34 mg pimavanserin (granulated) (equivalent to 40 mg pimavanserin tartrate), microcrystalline cellulose, such as microcrystalline cellulose having a particle size distribution (D90) of 180-340 m, for example Avicel PH302 or an equivalent microcrystalline cellulose, and/or magnesium stearate, for example vegetable grade are encapsulated in a capsule of size 4, for example a two-piece capsule.

Provided are also embodiments wherein 10 or 20 mg pimavanserin (granulated), microcrystalline cellulose, for example Avicel PH302 or an equivalent microcrystalline cellulose, and/or magnesium stearate, for example vegetable grade are encapsulated in a capsule of size 4, for example a two-piece capsule.

Provided are also embodiments wherein 34 mg pimavanserin (granulated), 59 mg microcrystalline cellulose, for example Avicel PH302 or an equivalent microcrystalline cellulose, and/or 1 mg magnesium stearate, for example vegetable grade are encapsulated in a capsule of size 4, for example a two-piece capsule. No other excipients were added.

Also provided is a pharmaceutical composition, comprising a capsule of pimavanserin and one or more pharmaceutically acceptable excipient(s) as provided herein, wherein the composition is formulated such that at least 80% of pimavanserin is released in 30 minutes upon administration to a subject.

Also provided is a pharmaceutical composition, comprising a capsule of pimavanserin and one or more pharmaceutically acceptable excipient(s) as provided herein, wherein the composition is formulated such that at least 80% of the pimavanserin is released from the composition within 30 minutes upon in vitro dissolution testing according to USP<711> (apparatus 1 (basket apparatus)).

In some embodiments, a pharmaceutically acceptable salt of pimavanserin is administered to the patient. In some specific embodiments, a tartrate salt of pimavanserin is administered to the patient.

In some embodiments, the pharmaceutically acceptable salt of pimavanserin comprises an anion selected from the group consisting of phosphate, sulphate, nitrate, diphosphate, besylate, bicarbonate, carbonate, clavulanate, edisylate, isothionate, borate, halide including e.g., chloride and bromide, nitrate, acetate, succinate, lactate, lactobionate, laurate, mandelate, malate, citrate, fumarate, maleate, oleate, oxalate, ascorbate, nicotinate, benzoate, mesylate, salicylate, stearate, tannate, tosylate, valerate, methanesulfonate, ethanesulfonate, benzenesulfonate, p-toluensulfonate, 2-ethane disulfonate, and naphthalenesulfonate.

The exact route of administration, dose, or frequency of administration would be readily determined by those skilled in the art and can be dependent on the age, weight, general physical condition, or other clinical symptoms specific to the patient to be treated.

In some embodiments, the tartrate salt of pimavanserin is administered daily. In some embodiments, the tartrate salt of pimavanserin is administered once daily. In some embodiments, the tartrate salt of pimavanserin is formulated for oral administration as a unit dose.

In an embodiment, pimavanserin is administered orally.

In an embodiment, pimavanserin is orally administered in a daily dose from about 0.5 mg to about 90 mg, or from about 8 mg to about 42 mg, or about 10 mg to about 60 mg.

In an embodiment, pimavanserin is orally administered in a daily dose from about 0.5 mg to about 120 mg, or from about 8 mg to about 42 mg, or about 10 mg to about 60 mg.

In certain embodiments, the above ranges are for pimavanserin free base. In certain embodiments, the above ranges are for a pharmaceutically acceptable salt, e.g., a tartrate salt of pimavanserin or any of the salts listed above.

In another embodiment, pimavanserin tartrate is orally administered in a daily dose of about 40 mg. In some embodiments, the daily dose is 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 80 mg, 88 mg or 102 mg. In an embodiment the daily dose of pimavanserin tartrate is administered once, twice or three times per day, for example a 40 mg dose of pimavanserin tartrate is administered once a day, or 20 mg pimavanserin tartrate is administered twice a day.

In another embodiment, pimavanserin is orally administered in a daily dose of about 34 mg. In some embodiments, the daily dose is 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 68 mg, 80 mg, 88 mg or 102 mg. In an embodiment the daily dose of pimavanserin is administered once, twice or three times per day, for example a 34 mg dose of pimavanserin is administered once a day, a 20 mg dose of pimavanserin once or twice a day, or 17 mg pimavanserin is administered twice a day, or a 10 mg dose pimavanserin is administered twice a day.

### Anti-Depressants

In certain embodiments in the methods provided herein, the subject is also administered a selective serotonin reuptake inhibitor (SSRI) or a serotonin-norepinephrine reuptake inhibitor (SNRI).

In some embodiments, SSRI or SNRI is selected from the group consisting of citalopram, desmethylcitalopram, didesmethylcitalopram, desvenlafaxine, escitalopram (LEXAPRO), fluoxetine (PROZAC), fluvoxamine, paroxetine (PAXIL), sertraline, vilazodone, vortioxetine, desvenlafaxine, duloxetine, levomilnacipran, sibutramine, tramadol, milnacipran, norsertraline, serproxetin, and venlafaxine.

### EXAMPLES

### Example 1: Relapse Prevention Study of Pimavanserin for the Treatment of Hallucinations and Delusions Associated with Dementia Related Psychosis

A double-blind, placebo-controlled study of the efficacy and safety of pimavanserin was conducted to evaluate relapse prevention in patients with dementia related psychosis. The primary endpoint of this study was time from randomization to relapse in the double-blind period. The secondary endpoint of this study was time from randomization to discontinuation from the double-blind period for any reason.

The protocol-defined relapse criteria for dementia related psychosis were: patient experienced a ≥30% increase (worsening) from Week 12 (double-blind Baseline) on the Scale for the Assessment of Positive Symptoms-Hallucinations+Delusions (SAPS-H+D) Total Score and had a Clinical Global Impression-Improvement (CGI-I) score of 6 (much worse) or 7 (very much worse), relative to the double-blind Baseline; patient was treated with an antipsychotic other than pimavanserin for dementia-related delusions and/or hallucinations; patient stopped study drug or withdrew from the study for lack of efficacy as reported by the patient or study partner/caregiver or the Investigator discontinued study drug due to lack of efficacy; or patient was hospitalized for worsening dementia related psychosis.

Exploratory endpoints of this study included change from double-blind Baseline to Week 38/End-of-Treatment (EOT) on the following: SAPS-H+D score; SAPS hallucinations domain score; SAPS delusions domain score; Clinical Global Impression-Severity (CGI-S) of dementia related psychosis; Zarit Burden Interview (ZBI) score; Karolinska Sleepiness Scale (KSS) score; EQ-5D-5L score; and CGI-I for dementia related psychosis from double-blind Baseline to Week 38/EOT.

Safety and tolerability of pimavanserin was also evaluated compared to placebo in patients with dementia related psychosis who have been stabilized on pimavanserin therapy. Safety endpoints were: treatment-emergent adverse events (TEAEs); serious adverse events (SAEs); withdrawals due to adverse events (AEs); clinically important changes in other safety assessments; Global Clinician Assessment of Suicidality (GCAS) score; Mini-Mental State Examination (MMSE) score; and Extrapyramidal Symptom Rating Scale (ESRS) score.

### Subject Selection

Male or female patients ≥50 and ≤90 years of age were eligible if they met the criteria for All-cause Dementia according to NIA-AA guidelines. Eligible patients met the clinical criteria for one of the following disorders, with or without cerebrovascular disease (CVD): Dementia associated with Parkinson's disease; Dementia with Lewy bodies; Possible or probable Alzheimer's disease; Frontotemporal degeneration spectrum disorders, including possible or probable behavioral variant frontotemporal dementia, progressive supranuclear palsy, or corticobasal degeneration; or Vascular dementia, including post-stroke dementia, multi-infarct dementia and/or subcortical ischemic vascular dementia (SIVD). Eligible patients also had an MMSE score ≥6 and ≤24 and has had psychotic symptoms for at least 2 months. In addition, eligible patients had all of the following scores at Visit 1 (Screening) and Visit 2 (open-label Baseline): SAPS-H+D total score ≥10; CGI-S ≥4 (moderately ill); and SAPS-H+D global item (H7 or D13) score ≥4 (marked). Patients with a known personal or family history or symptoms of long QT syndrome were excluded.

### Study Design

The study consisted of a screening period of up to 4 weeks (3-28 days); an open-label period of 12 weeks; a double-blind period of up to 26 weeks; and an approximately 4-week safety follow-up period.

During the open-label period, patients started pimavanserin 34 mg once daily (QD) by mouth beginning at Week 0 (open-label Baseline). Pimavanserin was continued daily at this dose level for at least the first week of treatment. After the first week, if the patient was unable to tolerate the 34 mg dose, the dose was decreased to 20 mg (provided as two 10 mg tablets) at any study visit (scheduled or unscheduled) until Week 4 (Visit 4). If the dose was decreased to 20 mg it may later be increased to 34 mg based on investigator judgment at any study visit (scheduled or unscheduled) until Week 4 (Visit 4). After Week 4 (Visit 4), the dose of study drug remained fixed at either 34 mg or 20 mg once daily.

To enter the double-blind period, eligible patients were required to meet the following response criteria at Weeks 8 and 12: patient experienced a ≥30% reduction (improvement) from Week 0 (open-label Baseline) on the SAPS-H+D Total Score and had a CGI-I score of 1 (very much improved) or 2 (much improved), relative to Week 0 (open-label Baseline). A patient who did not meet the response criteria at Week 8 and 12 were withdrawn from study drug and entered the safety follow-up period of the study.

Randomization occurred at the double-blind Baseline visit (Week 12). Patients were randomly assigned 1:1 to continue their pimavanserin dose (34 mg or 20 mg) or matching placebo. Randomization was stratified by most likely dementia subtype or most prominent cause of dementia (Alzheimer's disease or frontotemporal dementia spectrum disorders, vascular dementia, or Parkinson's disease dementia or dementia with Lewy bodies) and by region.

Relapse criteria were assessed weekly for the first 2 weeks after randomization (Weeks 13 and 14), every 2 weeks until Week 26, and every 4 weeks through Week 38. Relapse criteria was also evaluated at unscheduled visits. Any patients who met any of the relapse criteria after randomization were withdrawn from study drug and entered the safety follow-up period of the study.

### Results

### Open-Label Period

Table 1 shows that most patients (>60%) demonstrated sustained response to pimavanserin in the open label period.

TABLE 1
Disposition in Open-Label Study

| Disposition in | Overall | AD | PDD | DLB | FTD | VaD |
|---|---|---|---|---|---|---|
| Open-Label | Number | N = 229 | N = 59 | N = 22 | N = 6 | N = 35 |
| (N = 351) | (%) | n(%) | n(%) | n(%) | n(%) | n(%) |
| Randomized to | 217 | 137 | 42 | 10 | 3 | 25 |
| Double-blind | (61.8) | (59.8) | (71.2) | (45.5) | (50) | (71.4) |
| Period | | | | | | |
| Early Termination | 134 | 92 | 17 | 12 | 3 | 10 |
| | (38.2) | (40.2) | (28.8) | (54.5) | (50) | (28.6) |
| Reason for Early | | | | | | |
| Termination | | | | | | |
| Lack of Response | 70 | 46 | 9 | 9 | 2 | 4 |
| in Open | (19.9) | (20.1) | (15.3) | (40.9) | (33.3) | (11.4) |
| Label | | | | | | |
| Adverse Event | 27 | 18 | 4 | 1 | 1 | 3 |
| | (7.7) | (7.9) | (6.8) | (4.5) | (16.7) | (8.6) |
| Death | 1 | — | 1 | — | — | — |
| | (0.3) | | (1.7) | | | |
| Other | 36 | 28 | 3 | 2 | — | 3 |
| | (10.3) | (12.2) | (5.1) | (9.1) | | (8.6) |

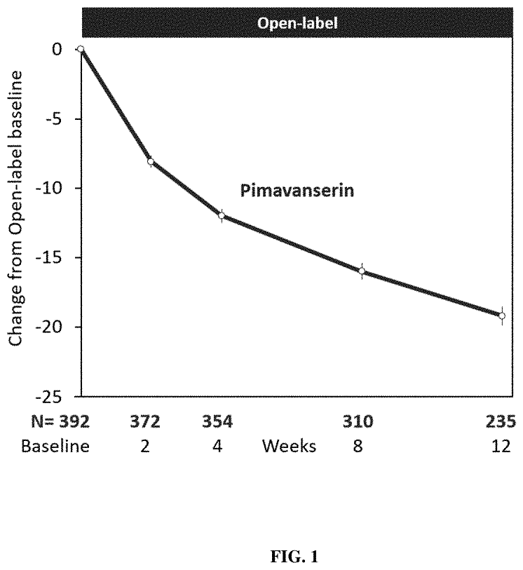

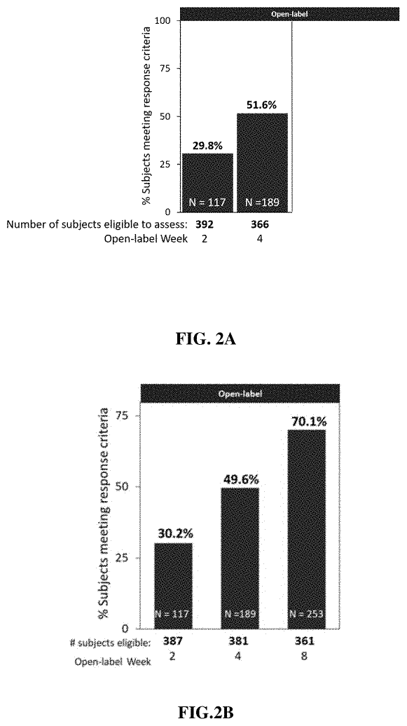

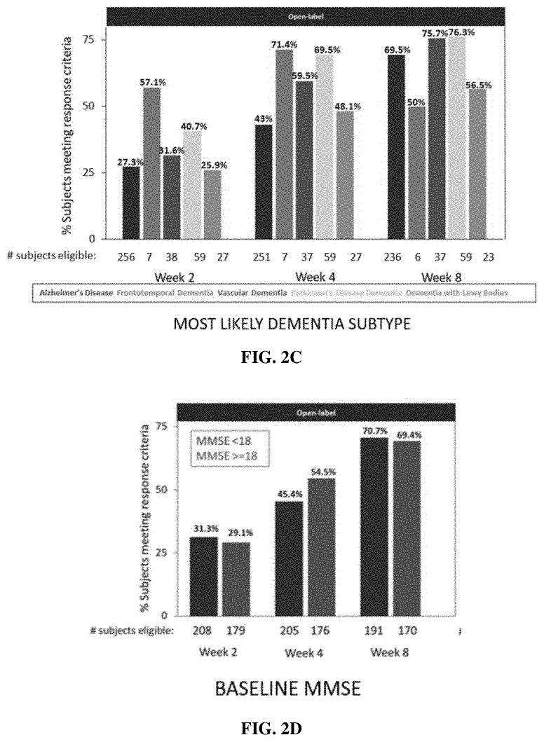

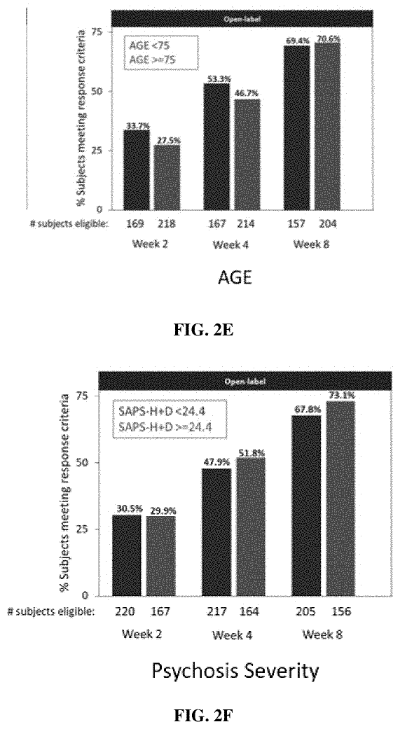

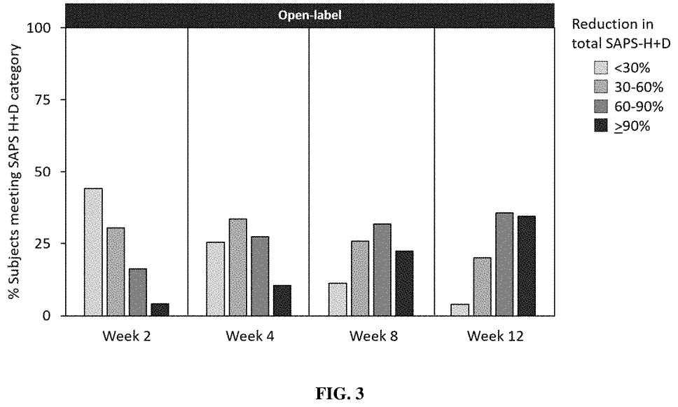

Based on the total SAPS-H+D change during the open-label period, it was demonstrated that pimavanserin substantially reduced psychosis symptoms. As shown in FIG. 1, 75.2% reduction in change from open-label baseline occurred by Week 12. In addition, pimavanserin treatment elicited a consistently high response rate. FIG. 2A shows that about 50% of patients met response criteria by Week 4. FIG. 2B further shows that about 70% of patients met response criteria by Week 8. Pimavanserin treatment triggered a consistently high response rate across subgroups including dementia subtypes (FIG. 2C), baseline MMSE (FIG. 2D), age (FIG. 2E), and psychosis severity (FIG. 2F). The response criteria was ≥30% reduction from open-label baseline on SAPS-H+D total score and CGI-I score of 1 or 2. Analysis was restricted to eligible patients at the visit. A patient was eligible for the visit if the number of days between minimum (study discontinuation date, end of treatment date) and the first open-label dose exceeded the lower limit of the analysis visit window for the visit. FIG. 3 also shows that pimavanserin treatment elicited a consistently high response rate.

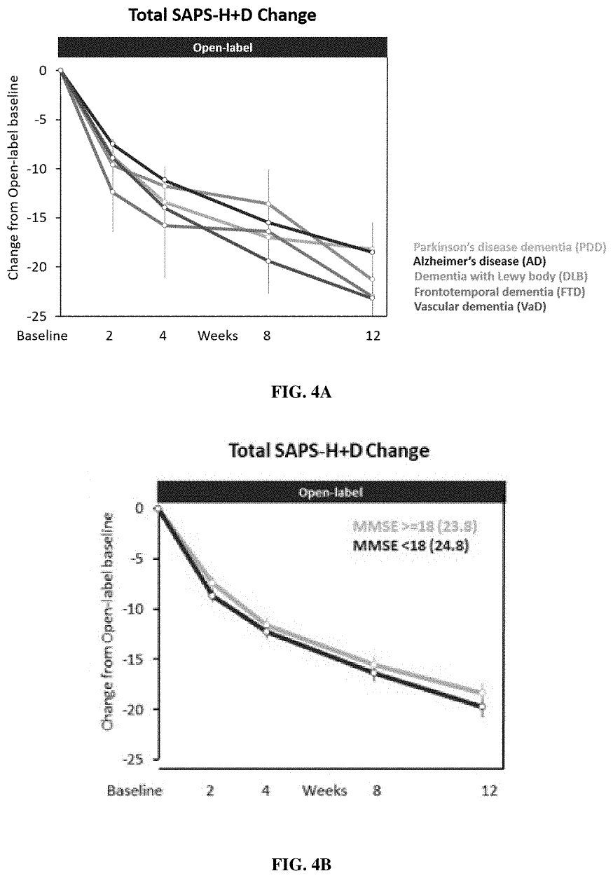

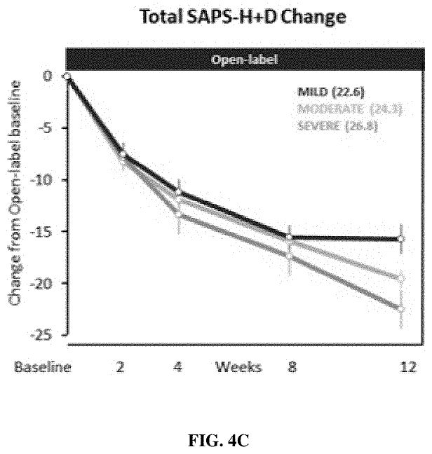

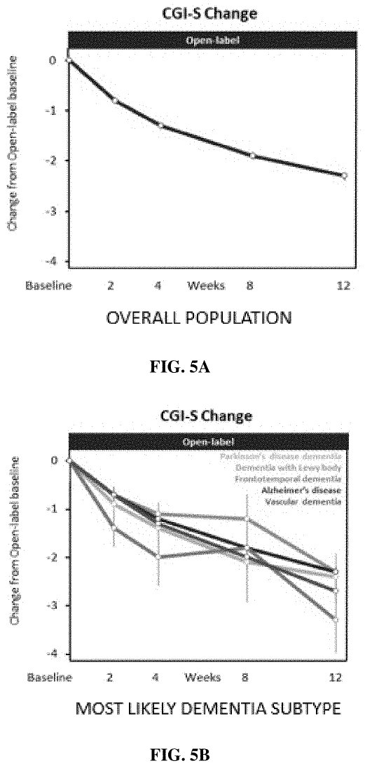

Furthermore, as shown in FIGS. 4A-C, exploratory analysis by most likely clinical diagnosis demonstrated that pimavanserin substantially reduced psychosis across subtypes of dementia (FIG. 4A), baseline MMSE (FIG. 4B), and baseline dementia severity (FIG. 4C). The CGI-S score in the patients treated with pimavanserin was reduced by about 2 from baseline by Week 12 for overall population (FIG. 5A) and across subtypes of dementia (FIG. 5B).

Based on the above, substantial reduction in psychosis symptoms was observed (by SAPS-H+D and CGI-S) with pimavanserin treatment irrespective of most likely clinical diagnosis of dementia subtype or baseline MMSE/dementia severity.

### Double-Blind Period

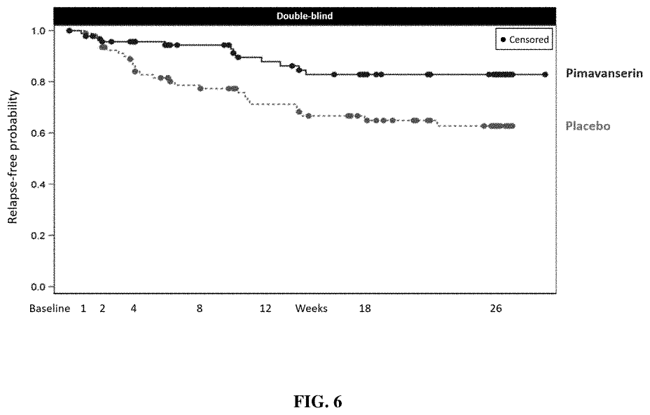

As shown in Table 2 and FIG. 6, pimavanserin significantly reduced risk of relapse of dementia related psychosis by 2.8 times compared to placebo.

TABLE 2
Patients having a relapsed event during the double-blind period

| | Placebo | Pimavanserin |
|---|---|---|
| | N = 99 | N = 95 |
| Number of Patients Having a Relapse | 28 (28.3) | 12 (12.6) |
| Event, n (%) | | |
| Number of Patients Censored, n (%) | 71 (71.7) | 83 (87.4) |
| Completed Week 26 Without a Relapse | 28 (28.3) | 37 (38.9) |
| Prematurely Discontinued Prior to Week 26 | 10 (10.1) | 9 (9.5) |
| Ongoing at Time of Database Cutoff | 33 (33.3) | 37 (38.9) |
| Cox Regression Analysis^{a} | | |
| Hazard Ratio (Pimavanserin/Placebo) | | 0.353 |
| 95% Confidence Interval | | (0.172, 0.727) |
| One-Sided p-value | | 0.0023 |
| O'Brien-Fleming Stopping Boundary | | 0.0033 |
| (p-value scale one-sided) | | |

^{a}The Cox regression model includes effects for treatment group, dementia subtype, and region.

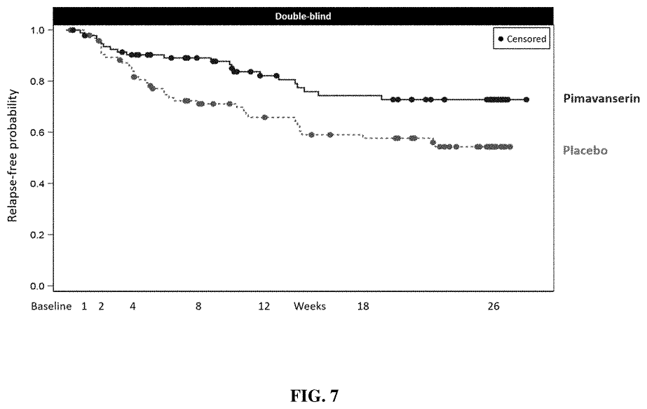

In addition, pimavanserin reduced risk of all-cause discontinuation by 2.2× compared to placebo (Table 2 and FIG. 7).

TABLE 3
Number of all-cause discontinuation during the double-blind period

| | Placebo | Pimavanserin |
|---|---|---|
| | N = 99 | N = 95 |
| Number of Patients Discontinued for | 38 (38.4) | 21 (22.1) |
| Any Reason, n (%) | | |
| Number of Patients Censored, n (%) | 61 (61.6) | 74 (77.9) |
| Completed Week 26 Without a Relapse | 28 (28.3) | 37 (38.9) |
| Ongoing at Time of Database Cutoff | 33 (33.3) | 37 (38.9) |
| Cox Regression Analysis | | |
| Hazard Ratio (Pimavanserin/Placebo) | | 0.452 |
| 95% Confidence Interval | | (0.261, 0.785) |
| One-Sided p-value | | 0.0024 |
| O'Brien-Fleming Stopping Boundary | | 0.0033 |
| (p-value scale one-sided) | | |

Table 4 shows the stabilization and relapse rates by most likely clinical diagnosis.

TABLE 4
Incidence of relapse by dementia subtype

| | Placebo | Pimavanserin | Hazard | Odds |
|---|---|---|---|---|
| | n/N (%) | n/N (%) | Ratio | ratio |
| Overall | 28/99 (28.3) | 12/95 (12.6) | 0.353 | 0.3666 |
| AD | 14/62 (22.6) | 8/61 (13.1) | 0.618 | 0.5175 |
| PDD | 10/20 (50.0) | 1/15 (6.7) | 0.054 | 0.0714 |
| DLB | 2/3 (66.7) | 0/6 (0.0) | — | — |
| FTD | 0/2 (0.0) | 1/1 (100) | — | — |
| VaD | 2/12 (16.7) | 2/12 (16.7) | 1.065 | 1.00 |

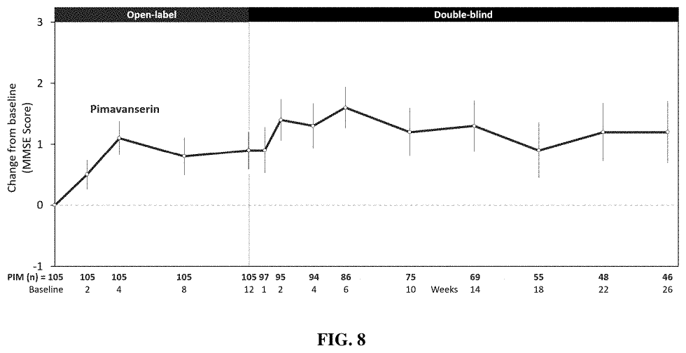

In addition, the study shows that there was no negative impact on MMSE over 9 months of treatment. FIG. 8 shows the MMSE score change from baseline through Week 46.

In the study, pimavanserin treatment showed significant maintenance of efficacy and response in dementia related psychosis. Pimavanserin reduced the risk of psychotic exacerbation by 2.8 times over placebo, and patients on placebo were 2.2 times more likely to drop out of the trial for any reason. Pimavanserin had no negative impact on a measure of cognition (MMSE) over 9 months of treatment. Moreover, pimavanserin 34 mg was well tolerated when given long term in the elderly population.

Although the invention has been described with reference to embodiments and examples, it should be understood that numerous and various modifications can be made without departing from the spirit of the invention. Accordingly, the invention is limited only by the following claims. All publications, patents, and patent applications cited in this specification are incorporated herein by reference as if each such publication, patent or patent application were specifically and individually indicated to be incorporated herein by reference.

### INCORPORATION BY REFERENCE

All publications and patents mentioned herein, including those items listed below, are hereby incorporated by reference in their entirety for all purposes as if each individual publication or patent was specifically and individually incorporated by reference. In case of conflict, the present application, including any definitions herein, will control.

### EQUIVALENTS

While specific embodiments of the subject disclosure have been discussed, the above specification is illustrative and not restrictive. Many variations of the disclosure will become apparent to those skilled in the art upon review of this specification. The full scope of the disclosure should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure.

What is claimed is:

1. A method of decreasing the risk of relapse in a patient having dementia related psychosis, comprising administering to the patient an effective amount of pimavanserin or a pharmaceutically acceptable salt, daily, wherein relapse is indicated by a) at least 15% increase of psychosis as measured by a SAPS-H+D Total Score in the patient and/or b) a CGI-I score of 3 or more after 12 weeks or more of administration of the pimavanserin.

2. The method of claim 1, wherein the relapse is indicated by a) at least 30% increase of psychosis as measured by a SAPS-H+D Total Score in the patient and/or b) a CGI-I score of 6 or more after 12 weeks or more of administration of the pimavanserin.

3. The method of claim 1 or 2, wherein the patient has moderate to severe psychosis before administration of pimavanserin, as measured by a SAPS-H+D total score of 10 more, a SAPS-H+D global item H7 or D13 score of 4 or more, and a CGI-S score of 4 or more.

4. The method of claim 1 or 2, wherein relapse is indicated by a) a 30% increase of psychosis as measured by a SAPS-H+D Total Score in the patient and b) a CGI-I score of 6 or more after 38 weeks of administration of the pimavanserin.

5. The method of any one of claims 1-3, wherein the patient has at least a 50% reduction in psychosis symptoms as measured by the SAPS-H+D Total Score at 12 weeks or more of administration as compared to psychosis symptoms before administration.

6. The method of any one of claims 1-5, wherein the patient has at least a 70% reduction in psychosis symptoms as measured by the SAPS-H+D Total Score at 12 weeks or more of administration as compared to psychosis symptoms before administration.

7. The method of any one of claims 1-6, wherein the risk of relapse is reduced by at least half.

8. The method of any one of claims 1-7, wherein the risk of relapse is reduced by at least 2.8 times.

9. The method of any one of claims 1-8, wherein the patient has at least at 30% reduction on SAPS-H+D total score and/or a CGI score of 1 or 2 after 4 weeks or more of administration as compared to a baseline before administration.

10. A method of treating dementia related psychosis in a patient in need thereof, comprising administering an effective amount of pimavanserin daily to the patient, wherein the patient has at least at 30% reduction on SAPS-H+D total score and/or a CGI score of 1 or 2 after 4 weeks or more of administration as compared to a baseline before administration.

11. The method of any one of claims 1-10, wherein after at least 4 weeks or more of administration, the risk of discontinuation of administration due to relapse or other adverse event is reduced by at least half compared to a patient not taking pimavanserin.

12. The method of any one of claims 1-11, wherein over 9 months or more of administration, the patient does not suffer from substantial cognitive impairment compared to before administration, as measured by a MMSE score.

13. The method of any one of claims 1-11, wherein after 9 months or more of administration, the patient does not suffer from substantial cognitive impairment compared to before administration, as measured by a MMSE score.

14. The method of any one of claims 1-13, wherein the dementia related psychosis is one of: Alzheimer's disease dementia, dementia with Lewy Bodies, Parkinson's disease dementia, vascular dementia, or frontotemporal dementia.

15. A method of treating psychosis associated with vascular or frontotemporal dementia in a patient in need thereof, comprising administered an effective amount of pimavanserin daily to the patient.

16. A method of decreasing the risk of relapse in a patient having dementia related psychosis, comprising administering to the patient an effective amount of pimavanserin or a pharmaceutically acceptable salt, daily, wherein relapse is indicated by a) a 30% increase of psychosis as measured by a SAPS-H+D Total Score in the patient and/or b) a CGI-I score of 6 or more after 12 weeks or more of administration of the pimavanserin.

17. The method of any one of claims 1-16, wherein the effective amount is a daily dose of pimavanserin of 20 mg, 34 mg or 40 mg.

18. The method of any one of claims 1-17, wherein the effective amount is a daily dose of pimavanserin of 34 mg, provided in one fixed dose or multiple fixed doses.

19. The method of any one of claims 1-16, wherein the patient is concomitantly taking a strong CYP3A4 inhibitor and the effective amount is a 10 mg daily dose of pimavanserin.

20. The method of claim 19, wherein CYP3A4 inhibitor is selected from the group consisting of ketoconazole, clarithromycin, indinavir, and itraconazole.

21. The method of any one of claims 1-20, wherein the pimavanserin is administered as a tartrate salt.

22. The method of any one of claims 1-21, wherein the patient is also administered a SSRI or a SNRI.

23. The method of claim 22, wherein SSRI or SNRI is selected from the group consisting of citalopram, desmethylcitalopram, didesmethylcitalopram, desvenlafaxine, escitalopram (LEXAPRO), fluoxetine (PROZAC), fluvoxamine, paroxetine (PAXIL), sertraline, vilazodone, vortioxetine, desvenlafaxine, duloxetine, levomilnacipran, sibutramine, tramadol, milnacipran, norsertraline, serproxetin, and venlafaxine.

24. The method of any one of claims 1-23, further comprising administering an anti-dementia agent to the patient.

25. The method of claim 24, wherein the anti-dementia agent is a cholinesterase inhibitor.

26. The method of claim 24, wherein the anti-dementia agent is selected from the group consisting of donepezil, galantamine, and rivastigmine.

27. A method of reducing the risk of exacerbation of psychosis secondary to neurodegenerative or vascular disorders in a patient in need thereof, comprising administering an effective amount of pimavanserin to the patient.

28. The method of claim 27, wherein the neurodegenerative or vascular disorder is Alzheimer's disease, dementia with Lewy Bodies, Parkinson's disease, vascular dementia, or frontotemporal dementia.

29. The method of any one of claims 26-28, wherein after 4 weeks or more of administration, the risk is reduced by at least half compared to a patient not taking pimavanserin.

30. A method of improving the response in a patient having dementia related psychosis, comprising administering to the patient an effective amount of pimavanserin or a pharmaceutically acceptable salt, once daily, wherein the reduction in total SAPS-H+D score in week 2 is better than before administration, and wherein the reduction in SAPS-H+D is maintained or further reduced in week 12.

31. A method of stabilizing a patient having dementia related psychosis, comprising administering to the patient an effective amount of pimavanserin or a pharmaceutically acceptable salt, once daily, wherein the reduction in total SAPS-H+D score in week 2 is better than before administration, and wherein the reduction in SAPS-H+D is maintained or further reduced in week 12.

32. The method of any one of claim 27-31, wherein the effective amount is a daily dose of pimavanserin of 20 mg, 34 mg, or 40 mg.

33. The method of any one of claims 27-32, wherein the effective amount is a daily dose of pimavanserin of 34 mg, provided in one fixed dose or multiple fixed doses.

34. The method of any one of claims 27-31, wherein the patient is concomitantly taking a strong CYP3A4 inhibitor and the effective amount is a 10 mg daily dose of pimavanserin.

35. The method of claim 34, wherein CYP3A4 inhibitor is selected from the group consisting of ketoconazole, clarithromycin, indinavir, and itraconazole.

36. The method of any one of claims 27-35, wherein the pimavanserin is administered as a tartrate salt.

37. A method of treating a patient with a recent history of psychosis, comprising administering an effective amount of pimavanserin to the patient.
